(19) United States
(12) Patent Application Publication
(10) Pub. No.: US 2012/0052135 A1
(43) Pub. Date: Mar. 1, 2012

(54) Phosphate Adsorbent

(75) Inventors: Gisela Witzel, Wustheuterode (DE); Peter O. Geisser, St. Gallen (CH); Erik Philipp, Wittenbach (CH)

(73) Assignee: VIFOR (INTERNATIONAL) AG, St. Gallen (CH)

(21) Appl. No.: 13/202,586

(22) PCT Filed: Mar. 1, 2010
(86) PCT No.: PCT/EP2010/052551

§ 371 Date: Nov. 14, 2011

(30) Foreign Application Priority Data
Mar. 2, 2009 (EP) 09154107.8

(51) Int. Cl. A61K 33/26; A61P 3/12

(52) U.S. Cl. 424/647; 424/646

(57) ABSTRACT

Subject of the present invention are compositions comprising a mixture of calcium, magnesium and iron salts for use as a pharmaceutical preparation for adsorbing phosphate, especially for use as pharmaceutical preparations for the treatment of hyperphosphataemia, chronic kidney deficiency as well as for the treatment of haemodialysis patients.

Subject of the present invention are compositions comprising a mixture of calcium, magnesium and iron salts for use as a pharmaceutical preparation for adsorbing phosphate, especially for use as pharmaceutical preparations for the treatment of hyperphosphataemia, for the treatment of chronic kidney deficiency (CKD) patients as well as for the treatment of haemodialysis patients. The compositions according to the present invention can be used in the treatment of human beings as well as in the field of veterinarian medicine.

It is well known that patients suffering from chronic kidney deficiency in most instances also suffer from a disorder in calcium- and phosphorous-self-regulation. Therefore as most frequently, concomitant disease in renal deficiencies renal osteopathy must be mentioned.

In renal osteopathy a decrease in intestinal calcium resorption followed by a decrease in calcium intercalation into bones leads to so called hypocalcaemia (acalcinosis) which finds its expression in mineralisation deficiencies and osteoporosis. Additionally in renal osteopathy insufficient phosphorous excretion can be noticed resulting in an increase of phosphorous levels in blood leading to hyperphosphataemia. The interaction of both phenomena manifests in secondary hyperparathyroidism leading to skeleton destruction.

Therefore in renal deficiencies such as especially chronic kidney diseases a careful control of phosphorous accumulation in the intestine and in blood or serum is necessary in order to prevent secondary hyperparathyroidism and metastatic calcification.

A common procedure in phosphorous reduction has to be seen in dietary phosphorous restriction which might be sufficient to control serum phosphorous levels in early stages of renal failure. In late stages or fatal renal failure and especially during long-term dialysis urinary excretion of phosphorous is usually minimal.

Additionally dietary restriction often can not guarantee a proper balance between phosphorous restriction and sufficient protein and mineral supply and therefore a balanced nutrition. Thus especially in advanced state of renal failure given pathological phosphorous levels can hardly be compensated.

As a consequence in the medical field administration of phosphate binding agents is widely practiced.

Well known phosphate binding agents are metal-ion containing compositions, mostly inorganic salts or metal ion containing polymers, e.g. Sevelamer in the form of mono-substances.

Very common phosphate binding adsorbents are based on aluminium containing salts or compositions such as aluminium hydroxide or aluminium hydroxycarbonate and other aluminium (III) compositions. One big drawback of such aluminium based phosphate adsorbents can be found in the partial solubility upon contact with gastric juice and the release of Al³⁺ in the stomach and the gastrointestinal tract. The toxic effects of Al³⁺ accumulation may in the long-run lead to encephalopathy.

As a substitute it has been found and generally accepted that calcium salts, e.g. calcium acetate and calcium carbonate, magnesium salts e.g. magnesium carbonate, lanthanum carbonate, iron compounds e.g. iron citrate, iron acetate, stabilised iron oxides, iron hydroxides, iron oxihydroxides or iron complexes, as described in U.S. Pat. No. 4,970,079, can bind phosphate. However the mentioned compounds or their ions can also be absorbed if the compounds are soluble or are solubilised in combination with food or with gastric juice. So e.g. hardly soluble salts such as the carbonates can react with the hydrochloric acid of the gastric juice and Ca²⁺ or Mg²⁺ can be formed. In case of iron compounds Fe³⁺ and further in combination with ascorbic acid Fe²⁺ can be formed. All these ions can be absorbed by physiological pathways.

Preparations for phosphate binding which are available on the market and described in the medical field normally consist of so called mono-preparations which provide the highest possible absorption of the used compounds often leading to an overdosage of the administered ions beyond the physiological need. Such overdosage may disturb the physiological balance and further strain the organism with additional side-effects due to such mineral overdosage. For example overdosage and resorption of high doses of calcium ions effect hypercalcaemia, large doses of magnesium cause hypermagnesaemia, accompanied from e.g. diarrhoea. Therefore the use as single agent of such preparations is limited.

The combination of more than one agent with phosphate binding capacity in a preparation for the treatment of hyperphosphataemia has been described for example in EP 1 046 410 A2 referring to the use of calcium- and magnesium-containing phosphate binding agents, which are characterized by the simultaneous application of calcium- and magnesium compounds which are easy soluble under physiological conditions. According to this invention the simultaneous administration is described to be beneficial as to the effect that the resorption of the calcium and magnesium ions is inhibited by the presence of each other.

Nevertheless the applied amount to effect sufficient phosphate adsorption has to be high and the inhibition effect is temporary so the risk of overdosage of calcium and magnesium remains.

Instead EP 0150792 discloses preparations containing calcium- and/or magnesium compounds which are hardly soluble under physiological conditions, which means pH 6 to 9, for the treatment of hyperphosphataemia. Such hardly soluble salts show solubility at low pH such as acid pH which can be found in the gastric juice. Therefore such compositions have to be administered in enteric-coated preparations to avoid solubilisation and resorption in the stomach.

EP 0 868 125 B1 refers to phosphate adsorbing compositions on the basis of iron(III)hydroxide stabilized with carbohydrates or humic acid which may additionally contain one or more calcium salts such as calcium acetate. Such calcium acetate addition is described to enhance phosphate binding capacity of the iron hydroxide compositions according to the invention especially with elevated pH such as a pH of more than 5. In order to achieve sufficient phosphate adsorption the amount of phosphate binding compounds such as iron hydroxide and calcium salts such as calcium acetate used in such preparations has to be high. Furthermore, the use of acetate in such compositions may lead to alkalosis.

Furthermore phosphate binding compositions containing a mixture of iron and calcium salts are known from DE 32 28 231 A1, which refers to a calcium salt on the basis of calcium-containing polymers especially from the group of calcium-containing polysaccharides wherein calcium ions are partially replaced by iron ions or other trace elements e.g. magnesium or zinc. The preparation of such doped polysaccharides is complex and salts of exactly defined ion ratios are not easy to achieve. Molar ratios or content of the physiologically relevant phosphate binding ions are not defined for such compositions.

Another composition for phosphate binding in the treatment of hyperphosphataemia is described by US 2004/0105896 referring to a so called “mixed metal compound” having a certain phosphate binding capacity, and comprising various metals, including lanthanum, cerium etc. According to one special embodiment the mixed metal compound may contain calcium, magnesium and iron ions in a predicted molar ratio of 3:3:2. The preparation of such mixed metal compound comprises co-precipitation of sulphate solutions of the intended metal ions under alkaline conditions. In such a precipitation process a chemical reaction between the co-precipitated compounds takes place which results in a co-precipitated compound containing the compounds bound to each other via chemical bonding. It is therefore obvious that such precipitation method constitutes a complex proceeding, too. Additionally it can be seen from the analysis of the effective ion contents that the predicted values can not be reached. In fact the above mentioned specific mixed metal compound, containing calcium, magnesium and iron, shows a measured Ca²⁺:Mg²⁺:Fe³⁺ ratio of 2.9:2.3:2. The preparation of precipitates with varying molar ratios of the ions calcium, magnesium and iron or a solution for preparing compositions containing the ions in the actually desired or predicted amount is not described. It appears that with the co-precipitation process only very limited molar ratios of the elements are achievable, bearing again the risk of overdosage of one of the elements. Furthermore such co-precipitates are described to show a highly pH dependent phosphate adsorption capacity. Additionally altered as well as dried precipitates show decreased adsorption capacity compared to unaltered and wet precipitates.

According to a scientific publication by the inventors M. Webb and N. B. Roberts of US 2004/0105896 in the Journal of Pharmaceutical Sciences (Vol, 91, No. 1, 2002, 53-66), mixed metal compounds in their experiments belong to the class of compounds known as mixed metal hydroxides, which are also referred to as “layered double hydroxides”, “hydrotalcitic materials” or “hydrotalcites”. It is well known that hydrotalcites are layered minerals, which are obviously totally different from a physical mixture or blend of powdered, particulate or granular metal salts.

Further mixed metal compounds, which are obtainable by co-precipitation of different metal compounds in alkaline solutions, are known from WO 2007/088343. In contrast to the above mentioned co-precipitates of US 2004/0105896, the mixed metal salts according to WO 2007/088343 only contain two different metal ions such as Fe-ions in combination with Mg- or Ca-ions, preferably Mg- and Fe-ions. Precipitates of Fe, Mg and Ca-ions are not described.

The aim of the present invention was to provide a composition with sufficient phosphate binding capacity for the daily recommended value taking the physiological absorption of its ingredients into account especially with respect to the minimisation of the absolute amount absorbed. Furthermore such composition should allow effective phosphate binding over a wide pH range without causing overdosage of the applied phosphate binding compounds and thus avoiding undesired side-effects.

Furthermore the process for production of such composition should be easy, reproducible and with reliable recovery rate and thus allow the preparation of compositions with exactly defined molar values. In addition such process should provide compositions with highly variable amounts of the relevant metal ions contained.

It was surprisingly found, that in the binding of phosphate under physiological conditions, e.g. for the treatment of hyperphosphataemia, for the treatment of CKD patients and/or for the treatment of haemodialysis patients the target of a good treatment regime without disturbing the physiological equilibriums by restricting the metal ion absorption to a physiologically acceptable amount, thus avoiding undesired side-effects due to overdosage, can be achieved by an optimal combination of calcium, magnesium and iron compounds. It has surprisingly turned out that such a combination allows a composition comprising a mixture of the relevant salts using only the recommended daily (dietary) allowances (RDA) and taking, in particular, into consideration the absorption ratio for iron under the condition of CKD and haemodialysis.

The inventor has acted on the assumption that an amount of 2000-3000 mg calcium in the form of calcium salts (e.g. acetate or carbonate) corresponds to the daily recommended amount of calcium salts for phosphate adsorption in the therapy of hyperphosphataemia. Furthermore an amount of 1000 mg magnesium corresponds to the daily recommended amount of magnesium carbonate for therapeutically phosphate adsorption.

As the recommended daily dietary intake to achieve a physiological calcium and magnesium absorption is only approximately one-third each of such therapeutically applied amounts, namely 800 mg calcium and 300 mg magnesium per day, such therapeutically applied higher amounts bear the potential of overdosage as already discussed. In addition, it has to be mentioned that the daily meals contain also calcium and magnesium, normally up to the RDA. Nevertheless the present invention allows that the total daily intake will not exceed about the double of the RDA values and will still be below the intake of using only a single calcium or magnesium phosphate binder. In elderly patients the amounts of calcium and magnesium ingested with the meals are lower, so the problem of overdose is less serious.

The inventor has now found that the recommended phosphate binding value or capacity can be achieved by combining calcium and magnesium in an amount according to the recommended daily intake, each exhibiting approximately one-third of the therapeutically needed phosphate binding value and complementing the remaining third with a third physiologically acceptable phosphate binding compound, chosen from the group of iron containing phosphate binding compounds. Surprisingly, with such a composition the recommended phosphate binding value can be achieved without overdosage of physiologically absorbable compounds contained.

Furthermore with such composition comprising a combination of several potent phosphate binding agents the invention provides a phosphate binding agent with improved efficacy characteristics especially with respect to enhanced phosphate binding capacity and decreased absorption of the applied compounds over a wide pH range.

Additionally the solution of mixing or blending several potent phosphate binding agents, especially in the form of their salts or as powders, in a physical mixture provides a manufacturing method of such compositions which can be easily and reproducibly carried out with high recovery rate. Such mixing or blending process is not bound to complex or elaborate process steps or careful reaction conditions. Furthermore the mere mixing of several salts or powders allows high variability in the resulting mixture with respect to the incorporated substances and their activity, which may even take into account the individual condition of a patient in need of a phosphate adsorber as described below. As especially iron compounds may differ widely in their phosphate binding capacity or activity the present invention provides a highly adaptable system with stable phosphate adsorption capacity despite such potential activity fluctuations of the varying compounds.

Furthermore by varying the composition and the amounts of the different components the final composition can easily be adopted to specific requirements in the treatment of hyperphosphatemic patients e.g. with respect to the grade of required phosphate adsorption, to additional calcium, magnesium or iron substitution or in accordance with the individual physical condition of the patient (e.g. its body weight, gender, age, pregnancy etc.).

None of the above cited documents discloses a physical blend or mixture of calcium, magnesium and iron salts for treatment of hyperphosphataemia or chronic kidney deficiency or for the treatment of haemodialysis patients. Furthermore a combination of the three salt components as provided by the present invention was not obvious from the existing state of the art. Those documents which describe mixtures of at least two phosphate binding salts such as EP 1 046 410 A2, EP 0 150 792 A2 or EP 0 868 125 B1 do not give any hint that it might be superior to add further phosphate binding components comprising an additional and different metal ion. Furthermore no hint can be found, that such combination of three different metal ion salts each providing a phosphate binding capacity per se, might on the one hand improve the phosphate binding capacity of such composition and at the same time allow minimization of the applied components to an amount according to the recommended daily dose allowances. Furthermore none of the documents offers the possibility of lowering the existing amounts to the recommended daily dosages and complementing the resulting lack in phosphate binding capacity by adding a third phosphate binding compound.

Compositions such as disclosed in DE 32 28 231 A1 and US 2004/0105896, which include all three metal ions only provide compositions obtainable via a complex reaction process of various metal salts in a limited range of accessible molar ratios. No information can be gained from such disclosure that the mere mixture or blending of inorganic salts of the relevant metal ions provides positive effects in phosphate binding, too. Furthermore neither DE 32 28 231 A1 nor US 2004/0105896 provides any information as to the possibility of reducing the amount of the included metal ions as to an amount according to the recommended daily doses. Whereas DE 32 28 231 A1 remains silent about metal ion contents or molar ratio of such components at all US 2004/0105896 only refers to one embodiment with a predicted molar ratio in the precipitate itself, which furthermore can not be achieved with the given reaction process. US 2004/0105896 remains silent about total amounts of metal ion contents to be applied or to any specific effects of different molar ratio contents. The molar ratio chosen in the composition according to US 2004/0105896 does not appear to result from any outstanding effects or special product properties and no reference is made as to such ratio with respect to the recommended daily dose allowances of the ions. Therefore the molar ratios shown have been chosen by chance.

Furthermore US 2004/0105896 does neither disclose the possibility of varying and balancing the complemented metal ion ratio nor does it offer the possibility to combine a wide variety of compounds and in any case maintain the phosphate binding capacity stable. Therewith US 2004/0105896 does certainly not offer the possibility of adjusting varying activities by balancing the composition of the single ingredients without a resulting lack in phosphate binding capacity.

It is therefore the object of the present invention to provide a composition comprising a mixture of calcium salt(s), magnesium salt(s) and iron salt(s) for use as a pharmaceutical preparation for adsorbing phosphate, which comprises adsorbing phosphate in the body and/or from body fluids, either internally within the metabolism pathway or externally e.g. from dialysates. It is especially object of the present invention to provide a composition comprising a mixture of calcium, magnesium and iron salts for use as a pharmaceutical preparation for the treatment of hyperphosphataemia, for the treatment of chronic kidney deficiency (CDK) patients and/or for the treatment of haemodialysis patients.

In the context of the present invention the term “salts” broadly refers to heteropolar compounds of positively charged calcium, magnesium or iron atoms and suitable negatively charged anions. Although the bond in such salts in general has essentially ionic character, the term “salt” includes also the possibility of the presence of more or less polar covalent bond shares, for example, in case of metal oxides or hydroxides, in particular, of iron.

The calcium and magnesium salts of such compositions can be selected from the group consisting of carbonates, hydrogen carbonates (bicarbonates), basic carbonates (comprising hydroxyl anions apart from carbonate), acetates, oxides, hydroxides, alginates, citrate, fumarate, gluconate, glutamate, lactate, malate, silicate, succinate, tartrate and mixtures thereof. It is preferred, that the calcium and magnesium salts of such compositions are selected from the group consisting of carbonates, hydrogen carbonates (bicarbonates), basic carbonates, acetates, oxides, hydroxides and mixtures thereof, more preferably the calcium and magnesium salts of such compositions are selected from the group consisting of carbonates and acetates and mixtures thereof. With respect to magnesium salts so called basic magnesium carbonates such as 4MgCO₃Mg(OH)₂5 H₂O, are especially preferred. A particularly preferred embodiment according to the invention comprises calcium carbonate (CaCO₃) and basic magnesium carbonate (such as 4 MgCO₃Mg(OH)₂5H₂O).

The iron salt of the composition according to the invention is preferably selected from the group consisting of iron oxide, iron hydroxide (Fe(OH)₃), iron oxihydroxide (sometimes referred to as FeO(OH), although the present invention intends to cover all iron(III)-oxy/hydroxyl compounds of varying water contents or condensation degrees), iron complex compounds and mixtures thereof. Preferably the iron salt is selected from iron(III)-salts. In a preferred embodiment the iron salt is selected from the group consisting of iron(III)-hydroxide and/or iron(III)-oxihydroxide and/or iron(III)-oxides and/or stabilized forms thereof. Preferably the iron salts are stabilized by carbohydrates and/or humic acid. Useful carbohydrates can be chosen from the group of mono-, di-, oligo- and/or polysaccharides. It is possible to stabilize such iron compounds using soluble or insoluble carbohydrates and/or mixtures thereof. As examples for such stabilizing carbohydrates starch, agarose, dextrane, dextrine, dextrane derivatives, cellulose and its derivatives, sucrose (saccharose), maltose, lactose or mannitol can be mentioned. Iron oxihydroxide salts stabilized by sucrose are particularly preferred. Such salts may contain additionally starch.

For example such stabilized iron oxihydroxide salts are described in EP 0 868 125 B1 or in WO 06/000547. Thus, the use of iron hydroxide or iron oxihydroxide preferably stabilized by carbohydrates and/or humic acid, more preferably stabilized by sucrose, is preferred because of the elevated adsorption capacity of such stabilized iron compounds compared to the capacity of non-stabilized iron compounds. Therefore the total amount of iron in the composition can be reduced.

A preferred composition according to the present invention comprises a physical mixture or blend of
calcium carbonate or calcium hydrogen carbonate (bicarbonate),
magnesium carbonate, basic magnesium carbonate (like 4MgCO₃Mg(OH)₂5H₂O) or magnesium hydrogen carbonate (bicarbonate), and
iron(III)-hydroxide and/or iron(III)-oxihydroxide and/or iron(III)-oxides and/or stabilized forms thereof, especially such forms which are stabilized by sucrose and optionally starch,
   preferably adjusting the molar ratios of the metals to the preferred ranges as defined herein, and preferably adjusting the daily dosages of the metals to the preferred ranges as defined herein.

As already pointed out the metal ions of the salts forming the phosphate binding composition are known to underlie physiological absorption in the stomach and the gastro intestinal tract, including the upper jejunum. Absorption thereby mainly depends on the solubility of the applied compound which is in most cases pH dependent. Therefore compounds which are easy soluble in acid pH are mainly absorbed in the stomach, especially before food uptake when the amount of gastric juice in the stomach is high. Compounds which are hardly soluble under acid condition but become soluble upon increase of pH will be absorbed in the intestine where the pH normally ranges between 5 to 8.

As already mentioned absorption of phosphate binding agents such as calcium, magnesium or iron ions may cause overdosage and thus malfunction, especially in compositions so far known and administered for phosphate binding.

It is general knowledge that iron from iron oxide (CAS Reg. No 1332-37-2) is sparingly absorbed and therefore iron oxides are generally recognized as safe (GRAS). Moreover the release and subsequently the absorption of Fe³⁺ from e.g. iron oxide is pH dependent. That means with higher pH only small amounts of Fe³⁺ are released from the iron salts. Accordingly Fe³⁺ will mainly be released and absorbed under acid conditions. Therefore the highest absorption will be under empty stomach conditions but not in combination with food as food uptake reduces gastric juice and therefore increases stomach pH.

The daily need of iron for a healthy adult is about 1 mg and will normally be absorbed from iron rich food (food containing 10-20 mg iron). Nevertheless patients suffering from chronic kidney deficiency and especially haemodialysis patients are limited in the absorption rate of iron by a factor of up to 10. Due to the chronic disease the synthesis of hepcidin, an iron absorption and iron metabolism blocker, in the liver is enhanced effecting a reduction of iron absorption. Additionally haemodialysis patients suffer from chronic blood loss and can therefore not be treated with oral iron preparation successfully. As even doses up to 200 mg of iron per day have to be applied, intravenous iron therapy is recommended in haemodialysis patients.

It is well known that the daily iron loss for haemodialysis patients is about 5 to 8 mg iron per day. The absorption rate from iron salts such as e.g. ferrous sulphate has been estimated to be approximately 1%. Therefore an amount of 500 to 800 mg iron from e.g. ferrous sulphate per day would be necessary to supply the recommended dose. But the application of such high doses of ferrous sulphate would lead to enormous incidence of gastro intestinal side-effects. Therefore in haemodialysis patient the intravenous iron therapy is the recommended standard. Nevertheless in CKD patients oral iron therapy is still used. Instead iron oxide is practically insoluble in the gastro intestinal tract especially in combination with food. Therefore for haemodialysis and CKD patients the applied intake of iron in form of iron oxihydroxide can be much higher than the recommended daily allowances as stated for healthy humans e.g. in “Richtlinie 90/496/EWG des Rates vom 24, Sep. 1990 über die Nährwertkennzeichnung von Lebensmitteln” or in US RDA (Recommended Dietary Allowance) and can be enhanced in such way that the finally absorbed iron does not exceed the amount of 1 mg which corresponds to that as recommended for healthy humans. 1 mg iron absorbed corresponds to a 5-10% absorption rate of the 14 mg value of the RDA.

The daily need of calcium is at about 800 mg, corresponding to 20 mmol Ca²⁺. Due to the fact that only about 30% of a dose of calcium compounds are absorbed the daily absorption is about 270 mg Ca corresponding to 7 mmol Ca²⁺. In case of hyperphosphataemia treatment calcium carbonate or calcium acetate are dosed daily up to 2000-3000 mg Ca²⁺. Such high doses lead to the well known side-effects of hypercalcaemia in haemodialysis patients. To avoid that type of side-effects calcium-free phosphate binders have been developed, e.g. lanthanum carbonate and sevelamer. These compounds however have the problem of not being physiological compounds. Although lanthanum is only sparingly absorbed it can be found in the bones. Sevelamer hydrochloride leads to acidosis. Additionally under lanthanum carbonate or sevelamer therapy not all patients absorbed enough calcium from the diet.

The daily need of magnesium is about 300 mg corresponding to 12.3 mmol Mg²⁺. In case of hyperphosphataemia treatment magnesium carbonate doses up to 465 mg Mg²⁺ have not shown the well known side effects as in case of higher doses, where diarrhoea and loose stools are reported. Nevertheless vascular calcification can be reduced by replacing calcium compounds against magnesium carbonate in hyperphosphataemia therapy.

In accordance therewith it is one main intention of the present invention to provide a composition with optimal phosphate binding capacity taking the physiological absorption rates and the daily recommended intake of the applied compounds into account, even with respect to absorption of iron under haemodialysis conditions.

The recommended daily dose allowance of calcium according to “Richtlinie 90/496/EWG des Rates vom 24, Sep. 1990 über die Nährwertkennzeichnung von Lebensmitteln” is 800 mg, corresponding to 20.0 mmol Ca²⁺.

The recommended daily dose allowance of magnesium according to “Richtlinie 90/496/EWG des Rates vom 24, Sep. 1990 über die Nährwertkennzeichnung von Lebensmilteln” is 300 mg, corresponding to 12.3 mmol Mg²⁺.

The recommended daily dose allowance of iron according to “Richtlinie 90/496/EWG des Rates vom 24, Sep. 1990 über die Nährwertkennzeichnung von Lebensmitteln” is 14 mg assuming an absorption rate of 5-10% (approximately 1 mg iron). As already mentioned absorption of iron is reduced by a factor more than 10, which would result in an allowed dose of at least 100 mg iron. However haemodialysis patients, but not CKD patients need approximately 5 mg iron per day because of daily blood loss in haemodialysis treatment. This higher need can be considered in assessing the possible higher daily dose of iron especially for haemodialysis patients, and therefore for patients suffering from hyperphosphataemia, without provoking iron overload. Furthermore there is also at least a factor of 10 between the absorption rate of iron from a soluble iron salt and practically insoluble iron oxihydroxide, which gives also security against iron overload in CKD patients. This results in a possible daily dose of at least 500 mg, corresponding to at least 9.0 mmol Fe³⁺.

It was surprisingly found that a composition comprising a mixture or blend of calcium, magnesium and iron salts, e.g. in form of a powder blend, can be administered in amounts up to the recommended daily dose allowance as defined above exhibiting optimal phosphate binding capacity without leading to metal ion overdosage and thus undesired side-effects.

Therefore a composition according to the present invention for administration of a mixture of calcium, magnesium and iron salts in a total amount based on the metal of

| | | |
|---|---|---|
| Ca²⁺: | 80 mg-2400 mg, | corresponding to 2-60 mmol |
| Mg²⁺: | 49 mg-729 mg, | corresponding to 2-30 mmol |
| Fe³⁺: | 112 mg-1676 mg, | corresponding to 2-30 mmol |

per daily dose can be provided.

Preferably a composition according to the present invention for administration of a mixture of calcium, magnesium and iron salts in a total amount based on the metal of

| | | |
|---|---|---|
| Ca²⁺: | 400 mg-1200 mg, | corresponding to 10-30 mmol |
| Mg²⁺: | 146 mg-439 mg, | corresponding to 6-18 mmol |
| Fe³⁺: | 279 mg-1117 mg, | corresponding to 5-20 mmol |

per daily dose is provided.

If the total amount of such compositions comprising the recommended daily dose of the calcium, magnesium and iron salts according to the above mentioned amounts is too high for administration in a single dose unit, the composition can be administered in several subsets or subunits per day. In one aspect of the present invention, the composition can therefore be administered in at least one (one or more) subsets or subunits per day. Furthermore the composition according to the present invention exhibits its phosphate binding capacity especially in combination with food uptake as one essential aspect of phosphate binding therapy has to be seen in binding of phosphate from food. Therefore the composition according to the present invention preferably has to be administered together with the meals.

Especially compositions according to the invention which are in the form of tablets, film tablets or capsules are limited in the amount which can be processed in such dosage form. Therefore it might happen, that such single unit dosage forms as tablets, film tablets or capsules do not contain the whole amount of one daily dose. Anyhow as the composition should preferably be administered together with the meals and thus in most of the cases have to be split over the day dosage forms containing only parts of the whole daily dose are preferred.

It is therefore preferred to administer the composition according to the invention in subsets for example by administering more than one tablet, film tablet, capsule either at once or split over the day. Such splitting over the day will be uncritical as long as per day the total amount of the recommended daily dose is achieved and as long as the composition of the mixture even in the sub units contains the molar ratio of the Ca²⁺, Mg²⁺ and Fe³⁺ ions as specified below. Nevertheless splitting of the daily dose into sub units is not restricted to compositions in the form of tablets, film tablets or capsules. In a particularly preferred embodiment the composition is in the form of a powder wherefrom several (more than one) smaller amounts or several (more than one) portions of the total daily dose amount will be administered split over the day together with each meal.

Therefore in one embodiment of the invention the total amount of the daily dose of the mixture of calcium, magnesium and iron salts is administered in several (more than one) subsets per day. Furthermore such subsets are for example in the form of a powder, a granule, capsules, tablets, film tablets, sachets or sticks. In another embodiment the composition according to the invention is administered in subsets wherein one subset comprises one quarter of the total amount per daily dose according to the ranges defined above.

For example, a combination of 800 mg (20 mmol) calcium (about ⅓ of the recommended daily dose for phosphate binding) with 300 mg magnesium (12 mmol) leads to absorption capacity of 32 mmol which is equivalent to 1300 mg calcium. This is about ⅔ of the above mentioned 2000 mg dose of calcium for phosphate binding. Furthermore a daily dose of about 7.5 g phosphate binder containing iron oxihydroxide (O Hergesell and E Ritz, Nephrology Dialysis Transplantation, Vol 14, Issue 4 863-867) corresponding to about 1500 mg iron leads to an decrease of serum phosphate. This means that in combination of calcium and magnesium this could be reduced to about ⅓ (500 mg iron, corresponding to 9.0 mmol iron). Taking an ordinary iron oxihydroxide with a lower phosphate binding capacity (e.g. ⅔ of that which was used by Hergesell) but with a higher iron content (e.g. 3 times higher) then 750 mg iron in form of 1190 mg iron oxihydroxide (Fe(OOH)) have to be used.

The composition according to the present invention can be varied by decreasing the calcium, magnesium or iron content to a minimum amount as given above compensating this decrease by increasing the remaining components to obtain steady phosphate binding capacity. Furthermore the composition can be varied by increasing the calcium and/or magnesium content in the ranges given above compensating a decrease in phosphate binding activity of iron compounds with reduced phosphate binding capacity to obtain steady phosphate binding values.

Nevertheless by varying the components the molar ratios have to be considered.

A composition according to the present invention contains preferably a molar ratio of Ca²⁺:Mg²⁺ from 1:0.02-20 and of Ca²⁺:Fe³⁺ from 1:0.02-20.

Also preferably a composition according to the present invention contains a molar ratio of Ca²⁺:Mg²⁺ from 1:0.20-0.78 or a molar ratio of Ca²⁺:Mg²⁺ from 1:0.80-0.99 or from 1:1.03-2.00

Another preferred composition according to the present invention contains a molar ratio of Ca²⁺:Fe³⁺ from 1:0.02-0.65 or a molar ratio of Ca²⁺:Fe³⁺ from 1:0.67-0.68 or from 1:0.7-0.99.

One particularly preferred embodiment according to the present invention contains Ca²⁺, Mg²⁺ and Fe³⁺ each in an amount up to the recommended daily dose allowance as defined herein.

Therefore such particularly preferred embodiment contains Ca²⁺, Mg²⁺ and Fe³⁺ in a total amount based on the metal of

| | | |
|---|---|---|
| Ca²⁺: | 800 mg, | corresponding to 20 mmol |
| Mg²⁺: | 300 mg, | corresponding to 12.3 mmol |
| Fe³⁺: | 500 mg, | corresponding to 9 mmol |

for administration per day, either in a single unit or in subsets administered at once or split over the day, preferably together with the meals.

The amount of iron compound of the composition according to the present invention depends on the phosphate binding capacity of the used iron compound. Especially the above named stabilized iron (III) compounds exhibit improved phosphate binding capacity and can therefore be administered in a lower total amount.

The phosphate binding capacity of e.g. the preferred compounds calcium carbonate, magnesium carbonate and iron oxides/hydroxides are pH dependent. Therefore with increasing pH the phosphate binding capacity of calcium and magnesium carbonate increases whereas the phosphate binding capacity or iron oxides/hydroxides decreases. Moreover the combination of carbonates with iron oxihydroxides guarantees a decreased iron solubility resulting in reduced iron absorption. This effect can be explained with respect to the immediate reaction of the carbonate with the acids in the gastrointestinal tract which further enhances the pH in the stomach. According to the solubility product of Fe(OH)₃ each increase of a pH unit decreases the solubility of iron by a factor 1000, what is enormous and influences the absorption of iron and the possible side effects definitively.

The pH-dependency of the compounds contained in the composition according to the present invention can be ranged as follows:

Calcium carbonate or hydrogen carbonate shows optimal phosphate binding capacity in weak acid pH. The binding capacity can be ranged: pH 3<pH 5.5>pH 8.

Magnesium carbonate, basic carbonate (such as 4 MgCO₃×Mg(OH)₂×5 H₂O) or hydrogen carbonate exhibits optimal phosphate binding capacity in neutral or weak basic pH such as under physiological condition in the intestine. The binding capacity can be ranged: pH 3<pH 5.5<pH 8.

Iron oxide/hydroxide shows optimal phosphate binding capacity in acid pH such as under physiological condition in gastric juice in the stomach. The binding capacity can be ranged: pH 3>pH 5.5>pH 8.

Furthermore the compounds applied with a composition according to the present invention prevent each other from being absorbed. Stabilized, insoluble iron hydroxide is enteral only sparingly absorbed as it enhances solubility under strong acid condition (<pH 3) only. The presence of carbonates prevents a decrease of the pH in the stomach below 3. Furthermore calcium inhibits absorption of iron and magnesium inhibits absorption of calcium and vice versa. Such mechanism further minimizes the risk of hypercalcaemia or hypermagnesaemia after application of the phosphate binding compound.

Therefore with the combination of the phosphate binding calcium, magnesium and iron salts according to the present invention a composition for treatment of hyperphosphataemia and chronic kidney deficiency can be provided which exhibits optimal and well balanced phosphate binding properties over a wide pH range between at least pH 2-8 as found under physiological conditions.

A further advantage of the composition according to the present invention can be seen in the easy and safe preparation method.

The compositions according to the present invention comprise a physical mixture or a blend of the salts. This means that the composition can be obtained by blending the calcium, magnesium and iron salts. Furthermore the composition can be obtained by blending powders, granules, crystals, crumbs or other available forms of calcium, magnesium and iron salts. Preferably the compositions are obtainable by blending powders of the salts.

Optionally the mixture of the calcium, magnesium and iron salts of the composition according to the present invention is a pressed mixed powder of the salts.

The composition according to the present invention can contain at least one further pharmaceutical substance and/or pharmaceutically acceptable excipient.

In one aspect of the invention the mixtures can be combined with further pharmaceutical substances which are especially needed in the treatment of patients suffering from hyperphosphataemia or chronic kidney deficiencies. Such additional pharmaceutical substances of interest are e.g. vitamin D and it's derivatives, antioxidants such as vitamin E and/or its derivatives, amino acids such as cystein, peptides such as glutathione, flavones and/or flavanoides or mixtures thereof.

In a preferred embodiment the composition according to the present invention contains at least one further pharmaceutical substance selected from vitamin D and/or its derivatives.

The mixtures according to the present invention can be provided as galenical formulations like e.g. capsules, tablets, film tablets, sachets, sticks, granules or powders. Such galenical formulations can be prepared in accordance with well known techniques using generally accepted excipients, auxiliary ingredients, colourants and flavours. Therefore the compositions according to the present invention are preferably in dry form.

Therefore in a further embodiment the composition according to the present invention contains at least one pharmaceutically acceptable excipient. Preferably such pharmaceutically acceptable excipient will be selected from the group of fillers, binder, colourants, flavours and/or ingredients for masking unpleasant tastes.

The compositions according to the present invention are for the treatment of humans as well as for the treatment of animals.

The composition according to the present invention is for oral or peroral administration, oral administration of the composition is preferred.

In one aspect of the invention the composition according to the present invention is a food supplement.

In another aspect of the invention the composition according to the present invention is administered in a time context with the food intake. In a further embodiment the composition according to the present invention is used by admixing the composition with at least one foodstuff. Such administration can be chosen irrespective of its use as food supplement or as pharmaceutical composition.

The previously described amounts of the salts of the composition which is subject to the present invention generally correspond to a mean normal daily dosage as defined herein which can be split into several (more than one) single doses, subsets or subunits to be taken with the daily meals. Preferably the daily dose is split into four parts comprising 2-times per day one part of the daily dose e.g. one for breakfast and one for dinner, and 2 parts for the main meal e.g. for lunch. It goes without saying that the dose can be split and administered in accordance with the individual nutrition intake behaviour of the patients. Altogether the splitting of the administered doses should be chosen in accordance with the amount, nutritional value and composition of each meal. For example phosphate rich meals e.g. meat and protein rich meals should be accompanied by higher doses. Nevertheless the daily recommended amount should preferably not be exceeded.

Therefore the present invention further comprises the use of the composition as defined herein wherein the administration of the total amount of the composition per daily dose according to the invention is split into subsets which are taken with each meal, wherein the total amount of the composition administered with the subsets per day constitutes the total daily amount according to the present invention.

Preferably the total amount of the composition per daily dose is split into four subsets each comprising one quarter of the total amount per daily dose according to the present invention and wherein two subsets are administered together with the main meal and one subset is administered together with two minor meals each.

The composition according to the present invention can be used for the preparation of a pharmaceutical composition for adsorbing phosphate, which comprises adsorbing phosphate in the body and/or from body fluids, either infernally within the metabolism pathway or externally e.g. from dialysates.

In the following preferred embodiments of the invention are summarized:
1. A composition comprising a mixture or a blend of calcium, magnesium and iron salts for use as a pharmaceutical preparation for adsorbing phosphate.
2. A composition according to embodiment 1, which comprises adsorbing phosphate in the body and/or from body fluids, either internally and/or externally.
3. A composition according to one of embodiments 1 or 2, comprising the treatment of hyperphosphataemia, the treatment of chronic kidney deficiency (CKD) patients and/or the treatment of haemodialysis patients.
4, A composition according to any of the previous embodiments, wherein the calcium and magnesium salts are selected from the group consisting of carbonates, hydrogen carbonates, basic carbonates, acetates, oxides, hydroxides and mixtures thereof.
5. A composition according to any of the previous embodiments, wherein the iron salt is selected from the group consisting of iron oxide, iron hydroxide, iron oxihydroxide, iron complex compounds and mixtures thereof.
6. A composition according to any of the previous embodiments, wherein the iron salt is selected from iron(III)-salts.
7. A composition according to any of the previous embodiments, wherein the iron salt is selected from iron(III)-hydroxide and/or iron(III)-oxihydroxide and/or iron(III)oxides and/or stabilized forms thereof.
8. A composition according to any of the previous embodiments, wherein the iron salts are stabilized by carbohydrates and/or humic acid.
9. A composition according to any of the previous embodiments, wherein the iron salts are stabilized by sucrose, optionally by sucrose and starch.
10. A composition according to any of the previous embodiments, wherein the molar ratio of calcium to magnesium is from 1:0.02-20 and the molar ratio of calcium to iron is from 1:0.02-20.
11. A composition according to embodiment 10, wherein the molar ratio of calcium to magnesium is from 1:0.20-0.78.
12, A composition according to c embodiment 10, wherein the molar ratio of calcium to magnesium is from 1:0.80-0.99.
13, A composition according to embodiment 10, wherein the molar ratio of calcium to magnesium is from 1:1.03-2.00.
14. A composition according to embodiment 10, wherein the molar ratio of calcium to iron is from 1:0.02-0.65.
15. A composition according to embodiment 10, wherein the molar ratio of calcium to iron is from 1:0.67-0.68.
16. A composition according to embodiment 10, wherein the molar ratio of calcium to iron is from 1:0.7-1.50.
17. A composition according to any of the previous embodiments for administration of a mixture of calcium, magnesium and iron salts in a total amount based on the metal of
   calcium: 80 mg-2400 mg, corresponding to 2-60 mmol
   magnesium: 49 mg-729 mg, corresponding to 2-30 mmol
   iron: 112 mg-1676 mg, corresponding to 2-30 mmol
   per daily dose.
18. A composition according to any of the previous embodiments for administration of a mixture of calcium, magnesium and iron salts in a total amount based on the metal of
   calcium: 400 mg-1200 mg, corresponding to 10-30 mmol
   magnesium: 146 mg-439 mg, corresponding to 6-18 mmol
   iron: 279 mg-1117 mg, corresponding to 5-20 mmol
   per daily dose.
19, A composition according to one of embodiments 17 or 18, wherein the total amount of the daily dose of the mixture of calcium, magnesium and iron salts is administered in one or more subsets per day.
20. A composition according to embodiment 19 wherein one subset comprises one quarter of the total amount per daily dose.
21. A composition according to any of the previous embodiments which comprises a mixture of
   calcium carbonate and/or calcium hydrogen carbonate,
   magnesium carbonate, magnesium hydrogen carbonate and/or basic magnesium carbonate, and
   iron(III)-hydroxide and/or iron(III)-oxihydroxide and/or iron(III) oxides and/or stabilized forms thereof.
22, A composition according to any of the previous embodiments which comprises a physical mixture or a powder blend, respectively, of the salts.
23, A composition according to any of the previous embodiments wherein the composition is obtainable by blending the salts,
24, A composition according to any of the previous embodiments, wherein the composition is obtainable by blending powders of the salts,
25. A composition according to any of the previous embodiments wherein the composition is an optionally pressed mixed powder of the salts.
26. A composition according to any of the previous embodiments containing at least one further pharmaceutically active substance and/or pharmaceutically acceptable excipient.
27. A composition according to embodiment 26, containing at least one further pharmaceutically active substance selected from vitamin D and/or its derivatives, antioxidants, like vitamin E and/or its derivatives, amino acids, like cystein, peptides, like glutathione, flavones and/or flavanoides or mixtures thereof.
28. A composition according to embodiment 26, containing at least one pharmaceutically acceptable excipient selected from the group of fillers, binder, colorants, flavours and/or ingredients for masking unpleasant tastes.
29. A composition according to one of the previous embodiments which is in the form of a powder, granules, capsules, tablets, film tablets, sticks or sachets,
30. A composition according to any of the previous embodiments which is for the treatment of humans,
31. A composition according to any of the previous embodiments which is for the treatment of animals.
32. A composition according to any of the previous embodiments which is for oral administration.
33. A composition according to any of the previous embodiments which is a food supplement.
34. A composition according to any of the previous embodiments which is for administration in a time context with the food intake.
35. Use of a composition as defined according to any of the previous embodiments for the preparation of a pharmaceutical composition for adsorbing phosphate in humans and/or animals,
36. Use of the composition as defined according to any of the previous embodiments wherein the composition is admixed with at least one foodstuff and/or further food supplement.
37. Use of the composition as defined according to any of the previous embodiments wherein the administration of the total amount of the composition per daily dose is split into subsets which are taken with each meal.
38. Use according to embodiment 37 wherein the total amount of the composition per daily dose is split into four subsets each comprising one quarter of the total amount per daily dose and wherein two subsets are administered together with the main meal and one subset is administered together with two minor meals each.
39. Use according to any of embodiments 35 to 38 wherein the total amount of the composition per daily dose is as defined in embodiments 17 or 18.

The present invention is illustrated by the following examples:

### EXAMPLES

The following examples constitute compositions for a daily dose each:

### Example 1

| | | Corresponding amount of metal |
|---|---|---|
| Compound | Amount | (Ca²⁺/Mg²⁺/Fe³⁺) |
| Calcium carbonate | 2000 mg | 20.0 mmol |
| Magnesium carbonate | 1037 mg | 12.3 mmol |
| Iron oxihydroxide* | 1191 mg | 13.4 mmol |
| Total | 4227 mg | |

*calculated as Fe(O)OH

From the composition of example 1 the following compositions can be deduced, substituting lower molar ratios of one component with higher ones of the other components.

### Example 2

| | | Corresponding amount of metal |
|---|---|---|
| Compound | Amount | (Ca²⁺/Mg²⁺/Fe³⁺) |
| Calcium carbonate | 1500 mg | 15.0 mmol |
| Magnesium carbonate | 1298 mg | 15.4 mmol |
| Iron oxihydroxide* | 1191 mg | 13.4 mmol |
| Total | 3989 mg | |

*calculated as Fe(O)OH

### Example 3

| | | Corresponding amount of metal |
|---|---|---|
| Compound | Amount | (Ca²⁺/Mg²⁺/Fe³⁺) |
| Calcium carbonate | 2500 mg | 25.0 mmol |
| Magnesium carbonate | 776 mg | 9.2 mmol |
| Iron oxihydroxide* | 1191 mg | 13.4 mmol |
| Total | 4466 mg | |

*calculated as Fe(O)OH

### Example 4

| | | Corresponding amount of metal |
|---|---|---|
| Compound | Amount | (Ca²⁺/Mg²⁺/Fe³⁺) |
| Calcium carbonate | 1000 mg | 10.0 mmol |
| Magnesium carbonate | 1560 mg | 18.5 mmol |
| Iron oxihydroxide* | 1191 mg | 13.4 mmol |
| Total | 3750 mg | |

*calculated as Fe(O)OH

### Example 5

| | | Corresponding amount of metal |
|---|---|---|
| Compound | Amount | (Ca²⁺/Mg²⁺/Fe³⁺) |
| Calcium carbonate | 2000 mg | 20.0 mmol |
| Magnesium carbonate | 1383 mg | 16.4 mmol |
| Iron oxihydroxide* | 800 mg | 9.0 mmol |
| Total | 4182 mg | |

*calculated as Fe(O)OH

### Example 6

| | | Corresponding amount of metal |
|---|---|---|
| Compound | Amount | (Ca²⁺/Mg²⁺/Fe³⁺) |
| Calcium carbonate | 2500 mg | 25.0 mmol |
| Magnesium carbonate | 1298 mg | 15.4 mmol |
| Iron oxihydroxide* | 595 mg | 6.7 mmol |
| Total | 4393 mg | |

*calculated as Fe(O)OH

### Example 7

In case of using a iron oxihydroxide with a 2 times lower phosphate binding capacity the composition is the following:

| | | Corresponding amount of metal |
|---|---|---|
| Compound | Amount | (Ca²⁺/Mg²⁺/Fe³⁺) |
| Calcium carbonate | 2500 mg | 25.0 mmol |
| Magnesium carbonate | 1298 mg | 15.4 mmol |
| Iron oxihydroxide* | 1191 mg | 13.4 mmol |
| Total | 4989 mg | |

*calculated as Fe(O)OH

Additionally the composition of example 1 can be changed by decreasing the calcium, magnesium or iron content to a minimum of e.g. 10-50% of that of example 1 and by compensation this decrease by increasing the remaining components to obtain the same phosphate binding capacity as in example 1.

Moreover in stead of carbonates also acetates can be used as far as alkalosis can be avoided.

Furthermore instead of an ordinary iron oxihydroxide a stabilised iron oxihydroxide as e.g. described in EP 0 868 125 B1 or U.S. Pat. No. 6,174,442 B1 can be used. Such iron oxihydroxides have the advantage of higher adsorption capacities. So the total iron dosage will be lower, e.g. instead of 750 mg only 500 mg, what will compensate the lower iron content of e.g. only 20-40% of such an ingredient. In the next examples such combinations comprising iron oxihydroxide stabilised by saccharose (sucrose) are compiled:

### Example 8

| | | Corresponding amount of metal |
|---|---|---|
| Compound | Amount | (Ca²⁺/Mg²⁺/Fe³⁺) |
| Calcium carbonate | 2000 mg | 20.0 mmol |
| Magnesium carbonate | 1037 mg | 12.3 mmol |
| Iron oxihydroxide* | 1523 mg | 9.0 mmol |
| stabilized** | | |
| (iron content 33%) | | |
| Total | 4560 mg | |

*calculated as Fe(O)OH
**stabilised by saccharose

### Example 9

| | | Corresponding amount of metal |
|---|---|---|
| Compound | Amount | (Ca²⁺/Mg²⁺/Fe³⁺) |
| Calcium carbonate | 1330 mg | 13.3 mmol |
| Magnesium carbonate | 1037 mg | 12.3 mmol |
| Iron oxihydroxide* | 2268 mg | 13.4 mmol |
| stabilized** | | |
| (iron content 33%) | | |
| Total | 4635 mg | |

*calculated as Fe(O)OH
**stabilised by saccharose

### Example 10

| | | Corresponding amount of metal |
|---|---|---|
| Compound | Amount | (Ca²⁺/Mg²⁺/Fe³⁺) |
| Calcium carbonate | 1670 mg | 16.7 mmol |
| Magnesium carbonate | 868 mg | 10.3 mmol |
| Iron oxihydroxide* | 2268 mg | 13.4 mmol |
| stabilized** | | |
| (iron content 33%) | | |
| Total | 4806 mg | |

*calculated as Fe(O)OH
**stabilised by saccharose

### Example 11

| | | Corresponding amount of metal |
|---|---|---|
| Compound | Amount | (Ca²⁺/Mg²⁺/Fe³⁺) |
| Calcium carbonate | 2000 mg | 20.0 mmol |
| Magnesium carbonate, | 1194 mg | 12.3 mmol |
| basic (4 MgCO₃ | | |
| Mg(OH)₂ 5 H₂O) | | |
| Iron oxihydroxide* | 1523 mg | 9.0 mmol |
| stabilized** | | |
| (iron content 33%) | | |
| Total | 4717 mg | |

*calculated as Fe(O)OH
**stabilised by saccharose

### Example 12

| | | Corresponding amount of metal |
|---|---|---|
| Compound | Amount | (Ca²⁺/Mg²⁺/Fe³⁺) |
| Calcium carbonate | 1330 mg | 13.3 mmol |
| Magnesium carbonate, | 1194 mg | 12.3 mmol |
| basic (4 MgCO₃ | | |
| Mg(OH)₂ 5 H₂O) | | |
| Iron oxihydroxide* | 2268 mg | 13.4 mmol |
| stabilized** | | |
| (iron content 33%) | | |
| Total | 4692 mg | |

*calculated as Fe(O)OH
**stabilised by saccharose

### Example 13

| | | Corresponding amount of metal |
|---|---|---|
| Compound | Amount | (Ca²⁺/Mg²⁺/Fe³⁺) |
| Calcium carbonate | 1670 mg | 16.7 mmol |
| Magnesium carbonate, | 1000 mg | 10.3 mmol |
| basic (4 MgCO₃ | | |
| Mg(OH)₂ 5 H₂O) | | |
| Iron oxihydroxide* | 2268 mg | 13.4 mmol |
| stabilized** | | |
| (iron content 33%) | | |
| Total | 4938 mg | |

*calculated as Fe(O)OH
**stabilised by saccharose

### Example 14

| | | Corresponding amount of metal |
|---|---|---|
| Compound | Amount | (Ca²⁺/Mg²⁺/Fe³⁺) |
| Calcium acetat x H₂O | 3163 mg | 20.0 mmol |
| Magnesium carbonate | 1037 mg | 12.3 mmol |
| Iron oxihydroxide* | 1191 mg | 13.4 mmol |
| Total | 5391 mg | |

*calculated as Fe(O)OH

### Example 15

| | | Corresponding amount of metal |
|---|---|---|
| Compound | Amount | (Ca²⁺/Mg²⁺/Fe³⁺) |
| Calcium acetat x H₂O | 3163 mg | 20.0 mmol |
| Magnesium carbonate, | 1194 mg | 12.3 mmol |
| basic (4 MgCO₃ | | |
| Mg(OH)₂ 5 H₂O) | | |
| Iron oxihydroxide* | 1523 mg | 9.0 mmol |
| stabilized** | | |
| (iron content 33%) | | |
| Total | 5881 mg | |

*calculated as Fe(O)OH
**stabilised by saccharose

The amounts mentioned in examples 1 to 15 correspond to a mean normal daily dosage which can be split in several single doses to be taken with the meals. Preferable the daily dose is split into four parts: 2-times one part for e.g. breakfast and dinner, and 2 parts for the main meal e.g. for lunch. All mixtures can be provided in form of galenical formulations like e.g. capsules, tablets, film tablets, sachets, granules and powders by using generally accepted excipients such as e.g. colourants and flavours. The mixtures can be combined with other substances for which a special or increased need exists in the treatment of patients suffering from hyperphosphataemia and/or chronic kidney deficiencies. Substances of interest are e.g. vitamin D and/or its derivatives, antioxidants, like vitamin E and/or its derivatives, amino acids, like cystein, peptides, like glutathione, flavones and/or flavanoides or mixtures thereof, etc.

### Example 16

### Investigation of the Effects of a Composition According to Example 11 on the Phosphorus-Availability in Cats

The phosphorus-binding capacity of a composition according to the present invention in the intestine of cats has been tested with regard to the reduction of phosphorus-uptake from food.

### Timing and Experimental Groups:

The investigation covered four experimental time-units each comprising 14 days, thus leading to a total time of the study of 4×2 weeks (8 weeks).

Experimental animal groups consisted of four groups of cats, each comprising two cats, wherein the animals had been selected taking into consideration the actual body measurements and the animal's sex. The average age of the cats was 2.5 years, and all animals were healthy and without any clinical conditions. Allocation of the dosage schedule to the groups was carried out at random. Each group of two animals was fed with a consistent dosage amount over the whole course of the experiment.

TABLE 1

| | | | Dosage¹⁾ of | Dosage of | |
|---|---|---|---|---|---|
| | | Initial Body | composition | composition | |
| Animal | Sex | Weight (BW) | 11/4 kg BW | 11/animal²⁾ | |
| 1 | female | 2162 g | 0 mg | 0 | mg |
| | | | (dosage I/control) | | |
| 2 | male | 4720 g | 0 mg | 0 | mg |
| | | | (dosage I/control) | | |
| 3 | male | 5368 g | 600 mg | 805.2 | mg |
| | | | (dosage II) | | |
| 4 | female | 3018 g | 600 mg | 452.7 | mg |
| | | | (dosage II) | | |
| 5 | female | 3166 g | 1200 mg | 949.8 | mg |
| | | | (dosage III) | | |
| 6 | male | 5824 g | 1200 mg | 1747.2 | mg |
| | | | (dosage III) | | |
| 7 | female | 3516 g | 1800 mg | 1582.2 | mg |
| | | | (dosage IV) | | |
| 8 | male | 6875 g | 1800 mg | 3093.75 | mg |
| | | | (dosage IV) | | |

¹⁾daily amount, administered in two food subunits per day
²⁾based on the initial body weight

An adaption phase of 2 weeks preceded the first experiment unit. In this adaption phase, no phosphate-binding composition was added to the cat's food.

In the following four experimental time-units, each of which was two weeks long, the cats received the composition according to example 11 mixed with their food according to the following dosage schedule:

TABLE 2

| | Time unit | Time unit | Time unit | Time unit |
|---|---|---|---|---|
| Dosage | 1 | 2 | 3 | 4 |
| I | Group 1 | Group 1 | Group 1 | Group 1 |
| II | Group 2 | Group 2 | Group 2 | Group 2 |
| III | Group 3 | Group 3 | Group 3 | Group 3 |
| IV | Group 4 | Group 4 | Group 4 | Group 4 |

### Nutrition:

The cats were fed with catfood with a comparatively low but covering demand of phosphorus according to table 3.

TABLE 3
Composition of the cat's food (%)

| | |
|---|---|
| Moisture in dry weight | 82.0% |
| Crude protein | 31.6% |
| Crude fat | 20.0% |
| Crude ash | 6.1% |
| Phosphorus | 0.5% |

Each cat was fed twice a day with an individual amount of food, calculated according to NRC 2006 (National Research Council 2006). The composition according to example 11 was mixed with each meal in the amount according to table 1.

### Results:

Body weight remained largely stable during the examination period. Health status remained unchanged.

Efficacy of the composition according to example 11 with regard to the phosphate-binding capacity from food was evaluated by:
food-uptake (g/day)
phosphorus uptake (mg/day)
urine volume (ml/day)
phosphorus concentration in the urine (mg/ml)
renal phosphorus excretion (mg/day)
renal phosphorus excretion/phosphorus uptake (%)

The following results/group were evaluated:

TABLE 4

| | Group 1 | | Group 2 | | Group 3 | | Group 4 | |
|---|---|---|---|---|---|---|---|---|
| | average | sd | average | sd | average | sd | average | sd |
| food-uptake | 126 | 11.10 | 152 | 5.3 | 185 | 35.48 | 131 | 8.2 |
| (g/day) | | | | | | | | |
| phosphorus | 115 | 10.07 | 138 | 4.8 | 168 | 32.18 | 119 | 7.4 |
| uptake (mg/day) | | | | | | | | |
| urine volume | 52 | 3.81 | 55 | 17.5 | 94 | 8.34 | 66 | 1.0 |
| (ml/day) | | | | | | | | |
| phosphorus | 0.72 | 0.01 | 0.55 | 0.2 | 0.44 | 0.13 | 0.25 | 0.1 |
| concentration | | | | | | | | |
| in the urine (mg/ml) | | | | | | | | |
| renal phosphorus | 37 | 2.14 | 25 | 0.3 | 41 | 14.38 | 15 | 5.9 |
| excretion (mg/day) | | | | | | | | |
| renal phosphorus | 33 | 1.26 | 19 | 0.9 | 25 | 3.65 | 13 | 5.9 |
| excretion/phosphorus | | | | | | | | |
| uptake (%) | | | | | | | | |

It became obvious that, as the dosage of the phosphate-binding composition according to example 11 increased, the phosphorus concentration in the urine (FIG. 1) and the renal phosphorus excretion (FIGS. 2 and 3) decreased. Food uptake was not influenced by the dosage amounts, which resulted in comparable phosphorus uptake throughout the groups.

Group 3 shows enhanced food and phosphorus uptake (FIGS. 4 and 5). Comparing the individual data of all animals according to table 5 (FIGS. 6 to 10), it becomes obvious that this results from a discrepancy in the data of animal no. 6.

TABLE 5

| | Group 1 | | Group 2 | | Group 3 | | Group 4 | |
|---|---|---|---|---|---|---|---|---|
| | Animal 1 | Animal 2 | Animal 3 | Animal 4 | Animal 5 | Animal 6 | Animal 7 | Animal 8 |
| food-uptake | 115.3 ± 9.1 | 137.5 ± 6.1 | 146.5 ± 30.1 | 157.0 ± 6.7 | 149.8 ± 23.3 | 220.7 ± 12.3 | 122.8 ± 22.1 | 139.2 ± 3.3 |
| (g/day) | | | | | | | | |
| phosphorus | 104.6 ± 8.2 | 124.7 ± 5.5 | 132.9 ± 27.3 | 142.4 ± 6.1 | 135.8 ± 21.1 | 200.2 ± 11.2 | 111.4 ± 20.1 | 123.2 ± 2.9 |
| uptake | | | | | | | | |
| (mg/day) | | | | | | | | |
| urine volume | 48.2 ± 5.2 | 55.8 ± 2.2 | 72.2 ± 10.5 | 37.1 ± 13.0 | 85.9 ± 9.7 | 102.6 ± 11.0 | 67.0 ± 10.6 | 65.0 ± 8.2 |
| (ml/day) | | | | | | | | |
| phosphorus | 0.7 ± 0.1 | 0.7 ± 0.1 | 0.4 ± 0.1 | 0.7 ± 0.2 | 0.3 ± 0.2 | 0.6 ± 0.3 | 0.3 ± 0.2 | 0.1 ± 0.0 |
| concentration | | | | | | | | |
| in the urine | | | | | | | | |
| (mg/ml) | | | | | | | | |
| renal | 35.3 ± 5.0 | 39.6 ± 5.0 | 24.9 ± 1.6 | 25.6 ± 7.9 | 26.1 ± 12.6 | 54.9 ± 21.6 | 21.3 ± 9.7 | 9.4 ± 2.9 |
| phosphorus | | | | | | | | |
| excretion | | | | | | | | |
| (mg/day) | | | | | | | | |
| renal | 34.3 ± 7.5 | 31.8 ± 5.0 | 19.9 ± 5.3 | 18.1 ± 6.5 | 21.0 ± 12.4 | 28.3 ± 13.0 | 19.3 ± 8.2 | 7.4 ± 2.1 |
| phosphorus | | | | | | | | |
| excretion/ | | | | | | | | |
| phosphorus | | | | | | | | |
| uptake (%) | | | | | | | | |

### Discussion:

The object of the investigation was to examine the phosphate-adsorbing efficacy of a composition according to the present invention.

Phosphorus adsorption in the intestine results in increased faecal and in decreased renal phosphorus excretion. This aspect is of importance especially in the treatment of patients suffering from renal insufficiency because, on the one hand, reduced renal phosphorus excretion means less stress on limited organ function and, on the other hand, thus counteracts hyperphosphataemia. As a result, the use of an effective phosphate-binder supports the treatment of patients with renal insufficiency.

The underlying investigation was able to show the efficacy of phosphate-binding compositions according to the present invention on the reduction of renal phosphorus excretion. Moreover, a dose-dependent effect could be observed by an increasing efficacy within the sense of a comparatively lower renal phosphorus excretion becoming visible as the dosage of the phosphate-binding composition increased (FIG. 9). Generally food-uptake was not influenced by enhanced dosage of the phosphate-binding composition and thus a comparable daily phosphorus uptake can be assumed. An exception has to be made with animal 6 from group 3 which showed higher-than-average food-uptake and thus higher-than-average phosphorus uptake and thus leading to deviant results in group 3 although, leaving animal 6 unconsidered, the described dose-dependant effects are clearly visible.

With respect to the deviating results of animal 6, it becomes obvious that individual conditions and influences may also have an impact. With the chosen study design such individual conditions are detectable, especially by grouping comparable test animals and by repeating the measurement cycles three times.

Finally, it can be stated that within one test group constant test results were achieved which show the efficacy of the phosphate-binding capacity.

Furthermore, with increasing dosage, increasing efficacy becomes obvious. As higher amounts of the phosphate-binding composition did not influence the food uptake, if can be assumed that comparable dosage-recommendations may lead to a remarkable reduction of renal phosphorus excretion, although even lower dosages of the phosphate-binding composition already reduced phosphorus excretion in the urine and thus exhibited efficacy. As a result, the applied dosage of the phosphate-binding composition the daily phosphorus uptake also has to be considered, because enhanced phosphorus uptake with food affords higher amounts of phosphate-binding composition for the effective reduction of renal phosphorus excretion. Daily phosphorus uptake is influenced by the nutrition, as well as by the individual food-uptake. Therefore the assessment of the efficacy of the phosphate-binding composition and the estimation of the dosage recommendation has to be evaluated on the basis of the daily phosphorus uptake. Thus, considering these aspects the phosphate-binding composition according to the underlying investigation seems to be suitable for reducing the phosphorus availability from food and thus, the renal phosphorus excretion in cats.

### FIGURES

FIG. 1:

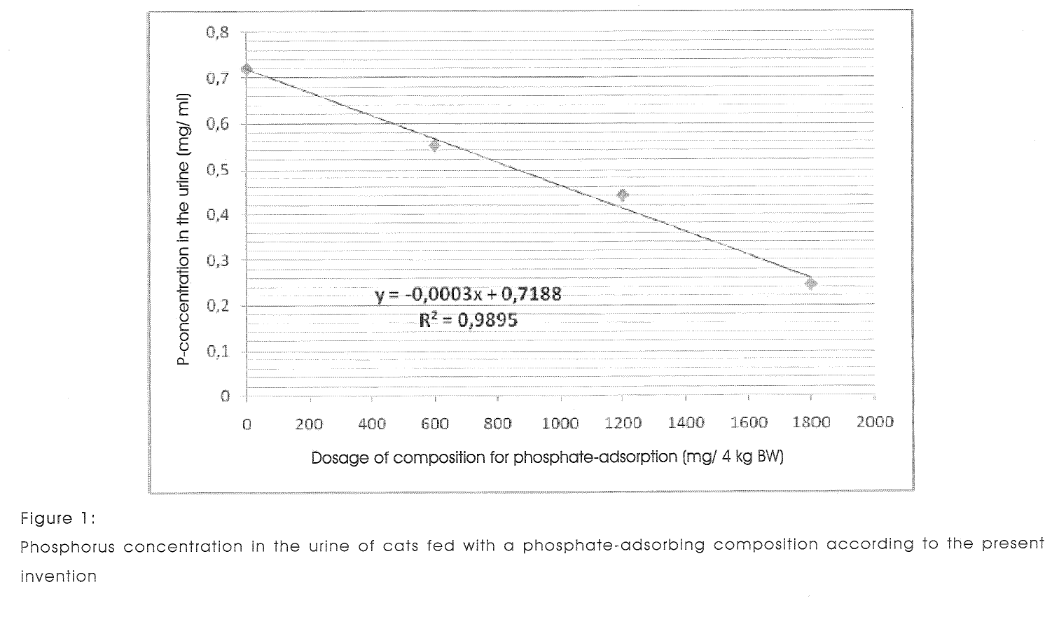

Phosphorus concentration in the urine of cats fed with a phosphate-adsorbing composition according to the present invention

FIG. 2:

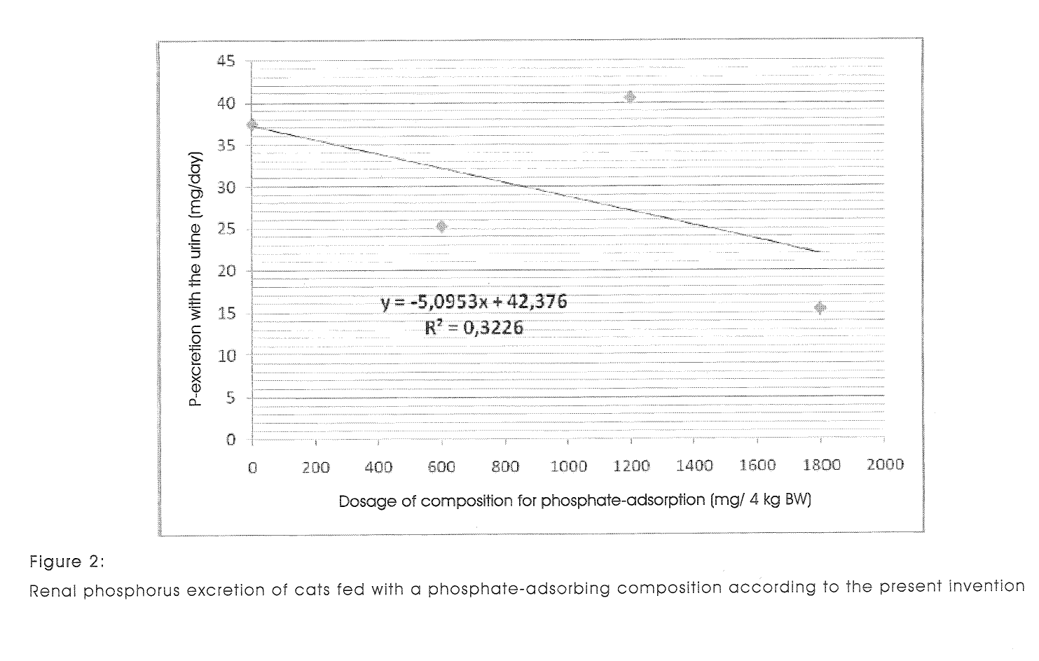

Renal phosphorus excretion of cats fed with a phosphate-adsorbing composition according to the present invention

FIG. 3:

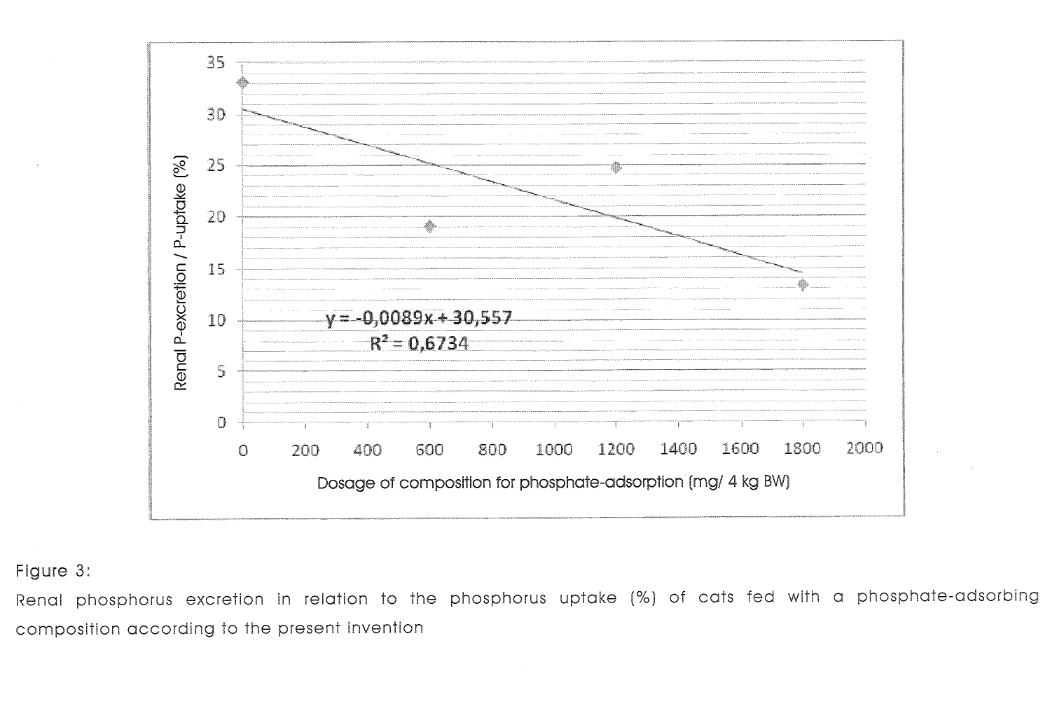

Renal phosphorus excretion in relation to the phosphorus uptake (%) of cats fed with a phosphate-adsorbing composition according to the present invention

FIG. 4:

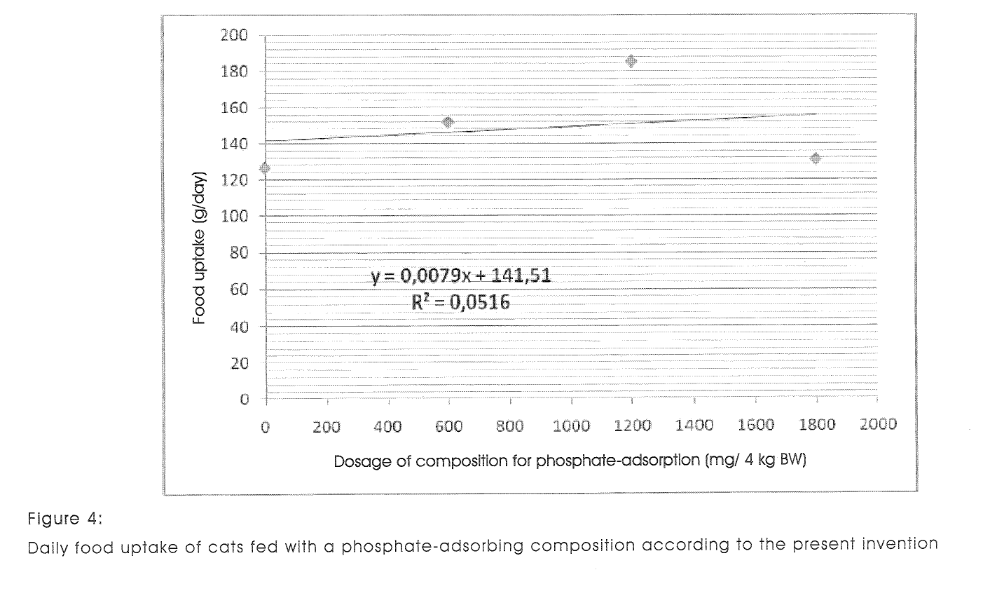

Daily food uptake of cats fed with a phosphate-adsorbing composition according to the present invention

FIG. 5:

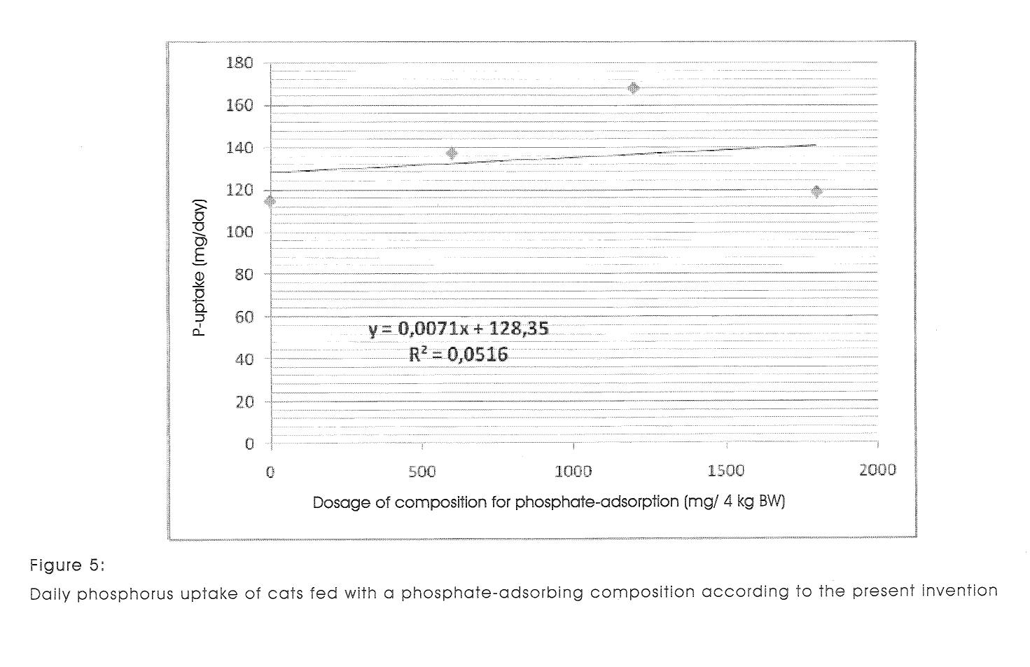

Daily phosphorus uptake of cats fed with a phosphate-adsorbing composition according to the present invention

FIG. 6:

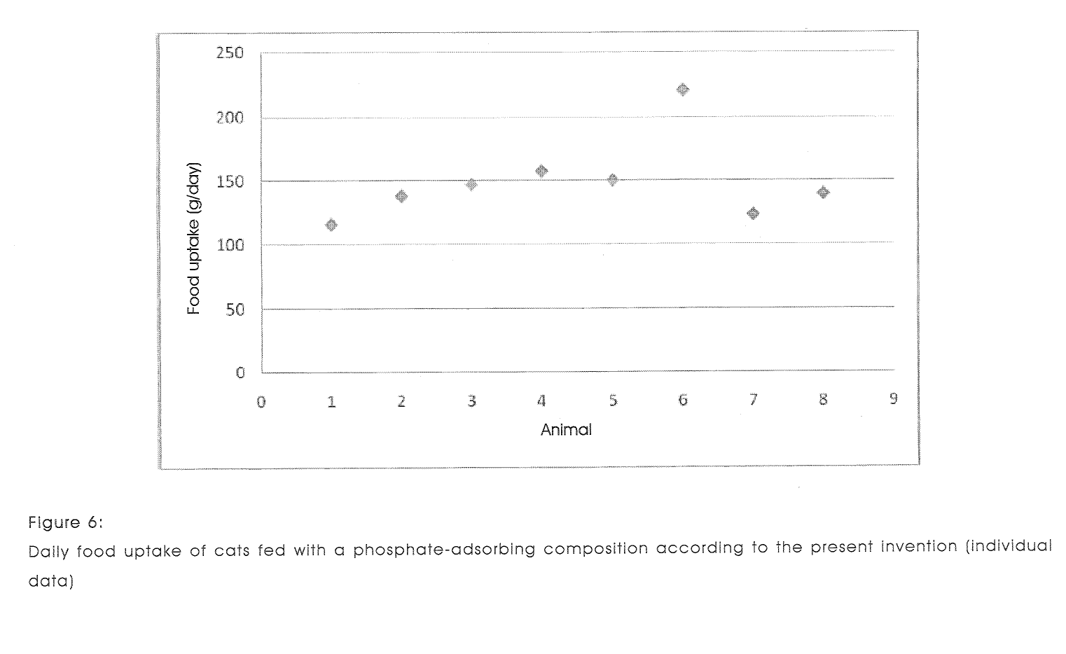

Daily food uptake of cats fed with a phosphate-adsorbing composition according to the present invention (individual data)

FIG. 7:

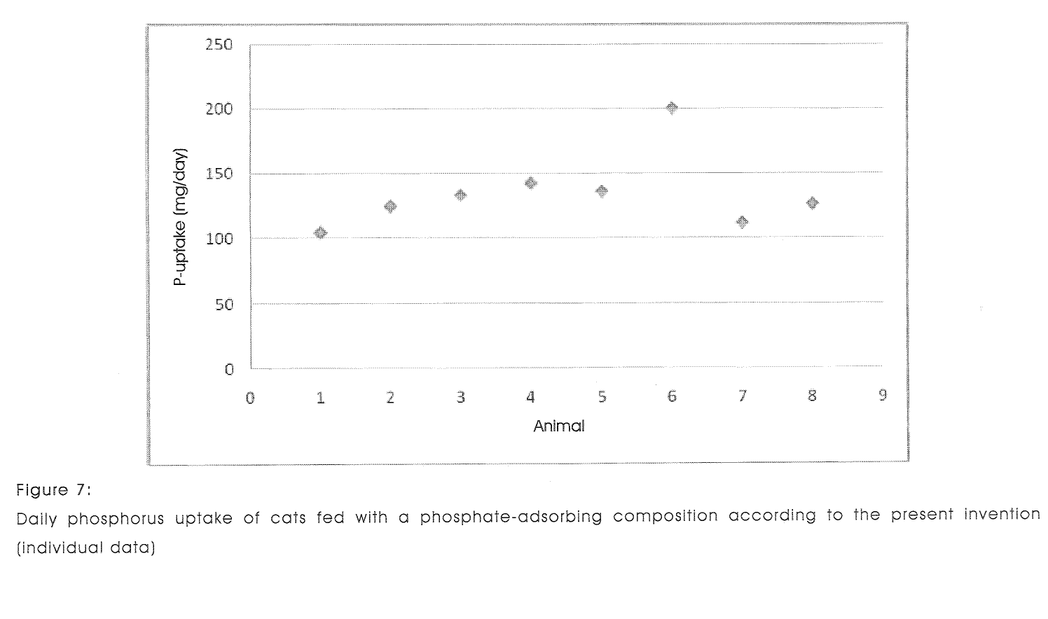

Daily phosphorus uptake of cats fed with a phosphate-adsorbing composition according to the present invention (individual data)

FIG. 8:

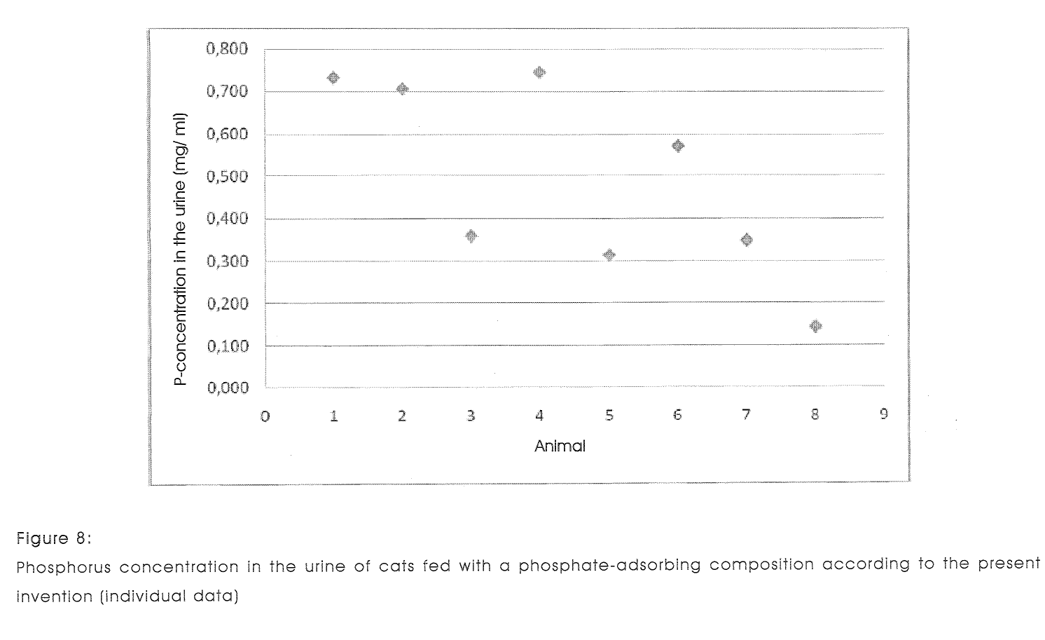

Phosphorus concentration in the urine of cats fed with a phosphate-adsorbing composition according to the present invention (individual data)

FIG. 9:

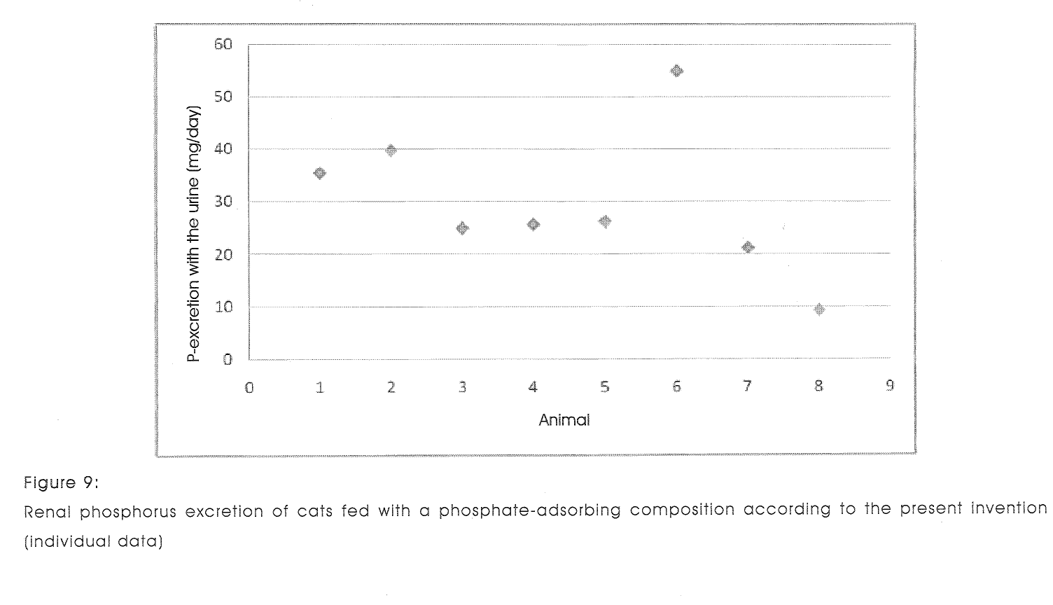

Renal phosphorus excretion of cats fed with a phosphate-adsorbing composition according to the present invention (individual data)

FIG. 10:

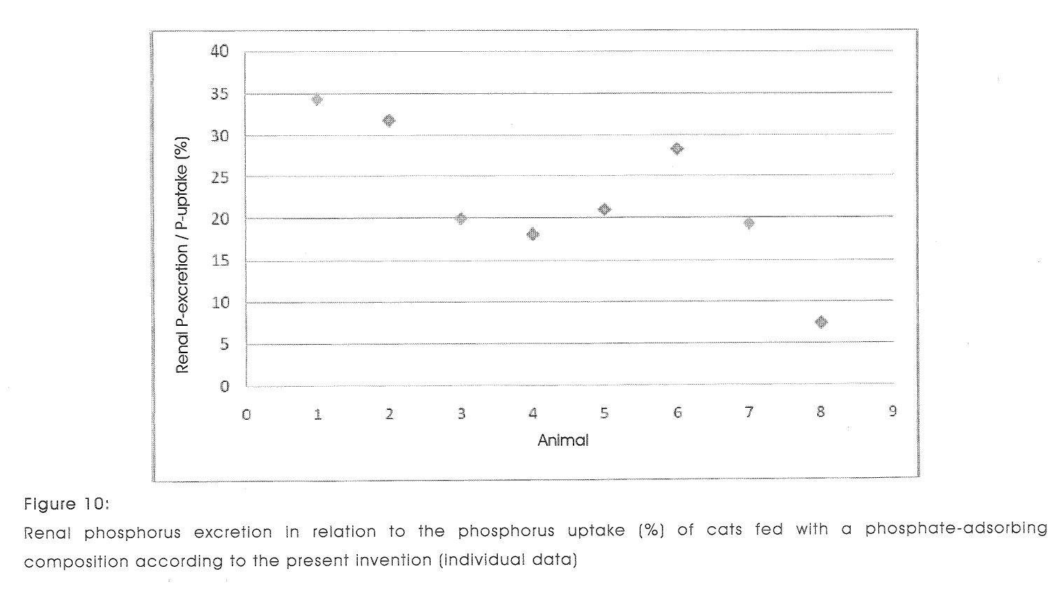

Renal phosphorus excretion in relation to the phosphorus uptake (%) of cats fed with a phosphate-adsorbing composition according to the present invention (individual data)

1. A composition comprising a mixture of calcium, magnesium and iron salts for use as a pharmaceutical preparation for adsorbing phosphate.

2-3. (canceled)

4. The composition according to claim 1, wherein the calcium and magnesium salts are selected from the group consisting of carbonates, hydrogen carbonates, basic carbonates, acetates, oxides, hydroxides and mixtures thereof.

5. The composition according to claim 1, wherein the iron salt is selected from the group consisting of iron oxide, iron hydroxide, iron oxihydroxide, iron complex compounds and mixtures thereof.

6. The composition according to claim 1, wherein the iron salt is selected from iron(III)-salts.

7. The composition according to claim 6, wherein the iron salt is selected from iron(III)-hydroxide and/or iron(III)-oxihydroxide and/or iron(III)-oxides and/or stabilized forms thereof.

8. The composition according to claim 1, wherein the iron salts are stabilized by at least one of carbohydrates and humic acid.

9. (canceled)

10. The composition according to claim 1, wherein the molar ratio of calcium to magnesium is from 1:0.02-20 and the molar ratio of calcium to iron is from 1:0.02-20.

11. The composition according to claim 10, wherein the molar ratio of calcium to magnesium is from 1:0.20-0.78.

12. The composition according to claim 10, wherein the molar ratio of calcium to magnesium is from 1:0.80-0.99.

13. The composition according to claim 10, wherein the molar ratio of calcium to magnesium is from 1:1.03-2.00.

14. The composition according to claim 10, wherein the molar ratio of calcium to iron is from 1:0.02-0.65.

15. The composition according to claim 10, wherein the molar ratio of calcium to iron is from 1:0.67-0.68.

16. The composition according to claim 10, wherein the molar ratio of calcium to iron is from 1:0.7-1.50.

17. The composition according claim 1 for administration of a mixture of calcium, magnesium and iron salts in a total amount based on the metal of
calcium: 80 mg-2400 mg, corresponding to 2-60 mmol
magnesium: 49 mg-729 mg, corresponding to 2-30 mmol
iron: 112 mg-1676 mg, corresponding to 2-30 mmol
per daily dose.

18-20. (canceled)

21. The composition according to claim 1, which comprises a mixture of
calcium carbonate and/or calcium hydrogen carbonate, magnesium carbonate, magnesium hydrogen carbonate and/or basic magnesium carbonate, and iron(III)-hydroxide and/or iron(III)-oxihydroxide and/or iron(III)-oxides and/or stabilized forms thereof.

22-25. (canceled)

26. The composition according to claim 1, containing at least one further pharmaceutically active substance and/or pharmaceutically acceptable excipient.

27. The composition according to claim 26, containing at least one further pharmaceutically active substance selected from the group consisting of vitamin D, derivatives of vitamin D, antioxidants, vitamin E, derivatives of vitamin E, amino acids, cystein, peptides, glutathione, flavones, flavanoides, and mixtures thereof.

28-29. (canceled)

30. The composition according to claim 1, which is for the treatment of humans.

31. The composition according to claim 1, which is for the treatment of animals.

33-34. (canceled)

35. A method of adsorbing phosphate in at least one of humans and animals comprising administering a pharmaceutical composition comprising the composition of claim 1 to at least one of humans and animals.

36-39. (canceled)
